(19) United States
(12) Patent Application Publication
(10) Pub. No.: US 2002/0151877 A1
(43) Pub. Date: Oct. 17, 2002

(54) MEDICAL LASER GUIDANCE APPARATUS

(76) Inventor: WILLIAM MASON, CAMBRIDGE (GB)

Correspondence Address: CANTOR COLBURN, LLP, 55 GRIFFIN ROAD SOUTH, BLOOMFIELD, CT, 06002

(21) Appl. No.: 09/214,286

(22) PCT Filed: Jun. 26, 1997
(86) PCT No.: PCT/GB97/01752

(30) Foreign Application Priority Data
Jul. 1, 1996 (GB) 9613766.6

(51) Int. Cl. A61B 18/20

(52) U.S. Cl. 606/4; 606/12

(57) ABSTRACT

An apparatus for applying laser light to the retina of the eye comprises a retina image obtaining means for obtaining the image of a retina. A retina image display displays the required retina image, and reference data receiving means receives, from an operator, data relating to a treatment to be performed. Template generating means generates a reference template on the retina image, and target position receiving means receives data related to at least one target point to which light is to be applied based upon the received reference data. Current retina position detecting means acquires a current retina image, and laser light application means directs and applies laser light to a retina.

This invention relates to a medical laser guidance apparatus.

In recent years laser light has been used to assist in the treatment of a number of ailments. One particular application of laser light has been in the treatment of eye disease by the application of laser light to the retina of an eye. Traditionally, such treatment has involved preliminary inspection of the eye using a fundus camera, slit lamp or similar optical viewing device followed by movement of the patient to a separate location for laser treatment of the eye. With such treatment, the laser is often applied days or weeks after the initial inspection, in view of the need to develop photographs taken by the fundus camera and the time required to analyse such photographs. Furthermore, once laser treatment is started, a number of sessions may be required, as the treatment is time consuming and traumatic for the patient, due to the need to compare the retina to be treated with earlier photographs. This, in turn, leads to the operator and patient tiring, making lengthy treatment sessions difficult to tolerate.

In order to overcome some of the above problems, it has been proposed to incorporate a laser treatment apparatus within a fundus camera. Such a system, whilst enabling the laser treatment of the retina to be performed at the same time as initial inspection, is extremely expensive in view of the large number of components required to provide a combined camera and laser application device. Furthermore, even with such systems it has been difficult to track a retina accurately throughout the inspection and treatment process, making the possibility of erroneous application of treatment light very high.

The present invention is directed towards solving some of the above problems.

According to the present invention there is provided an apparatus for applying laser light to at least one selected location on the retina of the eye, the apparatus comprising:

retina image obtaining means for obtaining the image of a retina;

retina image display means for displaying the required retina image;

reference data receiving means for receiving, from an operator, data relating to a treatment to be performed;

template generating means for generating a reference template on the retina image;

target position receiving means for receiving data related to at least one target point to which light is to be applied based upon the received reference data;

current retina position detecting means for acquiring a current retina image from the retina image acquisition means and comparing it to the generated template and said reference data and outputting a signal indicating current retina position within said template; and

laser light application means for directing and applying laser light to a retina dependent upon the output of the current retina position detecting means and the target position receiving means.

The reference data may include data relating to at least one reference point selected by an operator within the retina image.

An operator can view the position of low level laser light emitted by the laser light source by viewing the retina through the eye piece of the fundus camera, slit lamp or similar viewing apparatus, and can apply a treatment level of laser light to a selected point or area of a patient's retina by appropriate application of commands to the control means.

Alternatively, a video camera may be attached to the output of the viewing apparatus. The video camera output can be displayed on a video monitor for ease of viewing.

With either arrangement points on the retina may be selected for laser treatment by simple operation of a mouse, trackball or the like with an output connected to the target receiving means. With such an arrangement, semi-automated control of the laser is also possible by providing real time or near real time image recognition means which receives selected point or area data from an operator and then tracks the retina using video data from a camera and outputs treatment level laser light as required. This arrangement has the advantage that it can compensate for movements of the retina by the patient at a speed and accuracy that is not within the ability of a human operator.

A diffractive optical element may be placed within the optical path of the guidance apparatus in order to produce, from a single primary laser beam, an array of secondary treatment beams, which can thus be used to speed up treatment over a selected area, in order to reduce treatment time and patient discomfort.

The laser light source may be a tube laser, solid state laser or a diode laser, which has the advantages of reduced weight, size, and power consumption. The laser light positioning means may be a galvanometer and mirror arrangement, or may include acousto-optical devices. The laser light positioning means may be employed to modulate the light emitted by the laser light source by directing it away from the viewing apparatus optical path in a pulsed manner. Alternatively the laser light source may be pulsed.

One example of apparatus according to the present invention will now be described with reference to the accompanying drawings, in which:

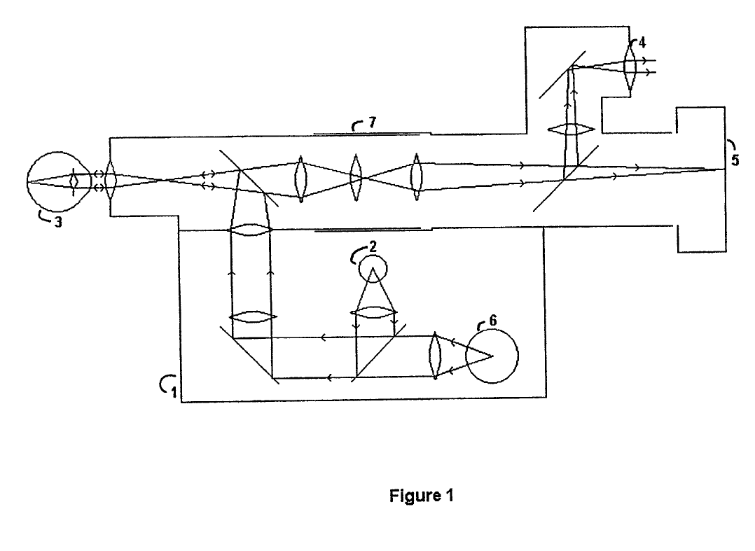

FIG. 1 is a schematic diagram of a known fundus camera;

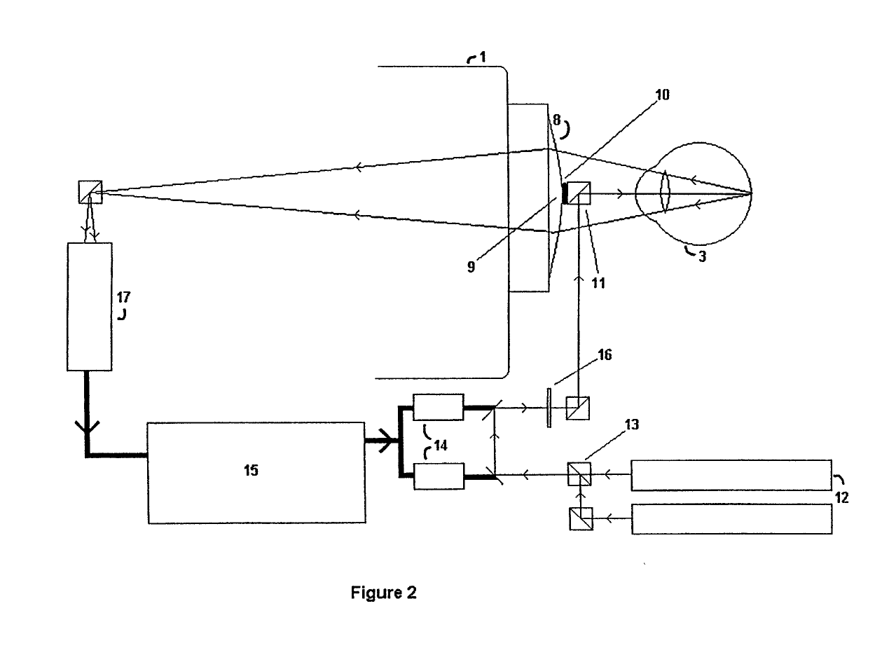

FIG. 2 is a schematic diagram of a guidance apparatus according to the present invention;

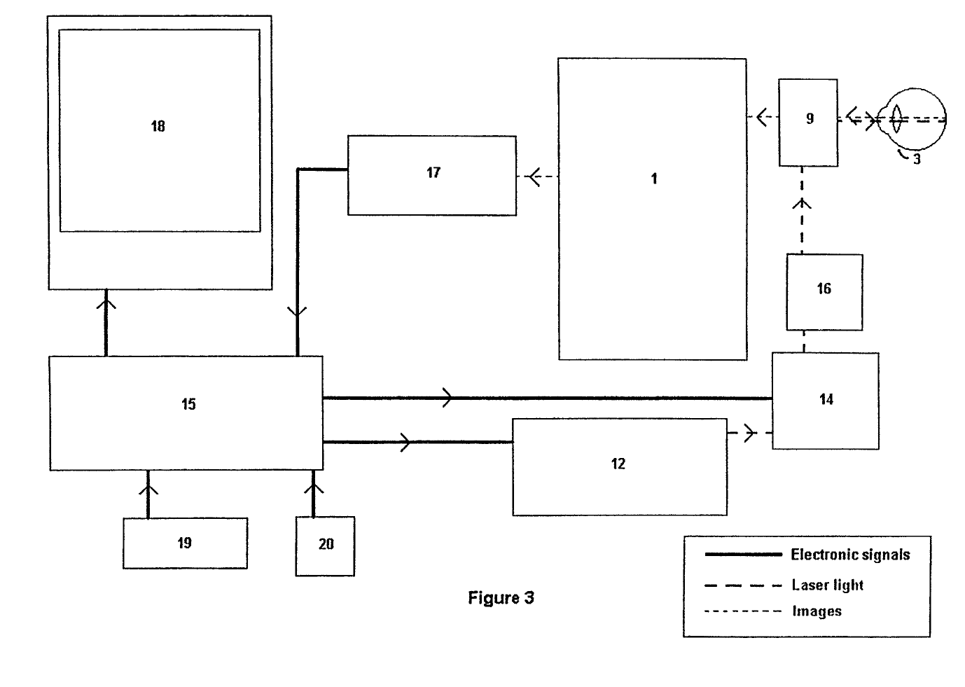

FIG. 3 is a schematic block diagram of a computer control guidance apparatus according to the present invention;

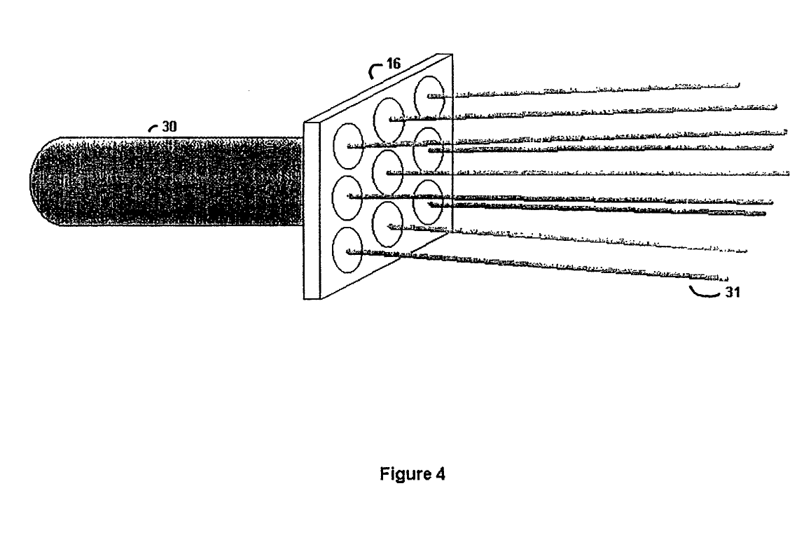

FIG. 4 is a diagram showing the operation of a diffractive optical element that may be employed in the present invention;

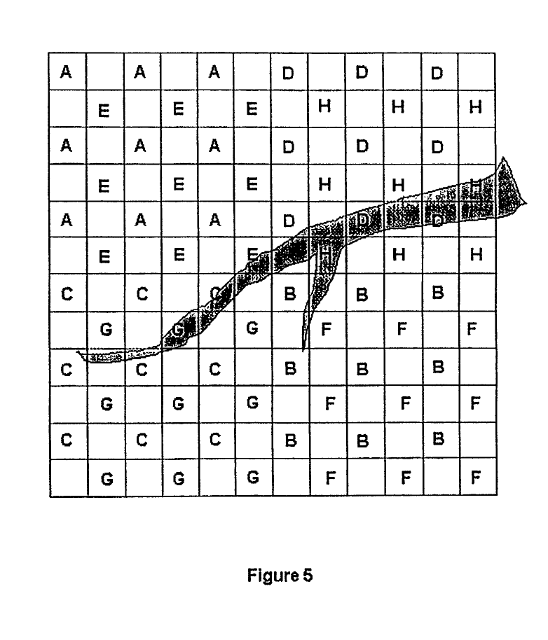

FIG. 5 is a diagram of a treatment template for an apparatus employing the diffractive optical element shown in FIG. 4; and,

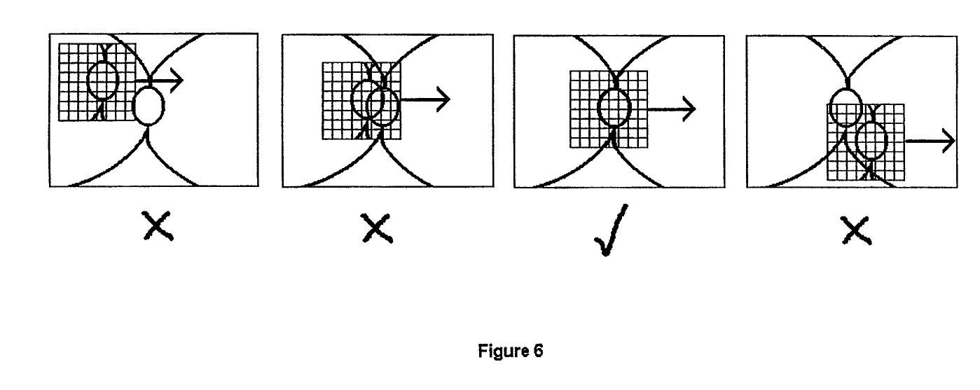

FIG. 6 is a schematic diagram showing an example image recognition technique employed with the present invention.

FIG. 1 shows a known fundus camera 1 which is employed to inspect the retina of an eye 3 which is to be treated. An illuminating light source 2 provides illuminating light to the eye 3, which light is then reflected back towards a viewing eyepiece 4 and camera 5. When a photograph of the retina is to be taken, a flash source 6 is activated and a photograph taken with the camera 5. A focusing system 7 is provided to focus the image of the retina for clear viewing by both the camera 5 and through the eyepiece 4.

FIG. 2 shows an example of the present invention being operated in conjunction with the known fundus camera 1 of FIG. 1. Corresponding components are identically numbered. It will be appreciated that this example could be adapted to operate in conjunction with a slit lamp or similar retina viewing apparatus. Attached to the objective lens 8 of the fundus camera 1 is a light directing element 9. The light directing element 9 comprises a circular beam stop 10 and prism 11. The prism 11 is arranged to direct light from a laser light source 12 into the optical path of the fundus camera 1 and on to the retina of the eye 3.

In this example, the laser light source 12 comprises a target laser light source 12 a and a main pulse laser light source 12b. It will be appreciated, however, that a single laser light source, operating at two different power levels could be employed. In this example, the target laser light source may be a HeNe or red diode laser, and the main pulse laser light source an argon or NdYg laser.

Light from the laser light source 12 is directed via a dichroic mirror 13 to a laser light positioning means 14 which is under the control of a laser light application means 15, in this example a computer, and comprises two X Y galvo mirrors. This arrangement may be substituted by an acousto-optical device placed in the light path. The light positioning means 14 operates via prisms to position light from the laser light source 12 within the optical path of the fundus camera 1 and to control the light's position on the retina of the eye. An optional diffractive element 16 is placed in the path of the light from the laser light source 12. The operation of this optional diffractive element 16 will be described later. It will be appreciated that many of these components could be placed within the camera housing if a dedicated apparatus were required.

Light from the laser light source 12 passes, in use, via the light directing means 9, on to the retina of the eye 3 and back into the fundus camera 1 via the objective lens 8. The light is then passed out, via the eyepiece 4 or a TV part (not shown) of the fundus camera 1, to a retina image receiving means 17, in this case a video camera 17. The output of the video camera 17 is, in this case, connected to a computer system.

Referring to FIG. 3, the overall operation of the apparatus according to the invention will now be described. Again, components corresponding to those described with reference to FIGS. 1 and 2 are identically numbered. In this example, a display means (e.g. a monitor) 18 is provided to receive outputs from the control means 15 and hence to display images received by the video camera 17. Reference data receiving means 19, 20 are provided, and may be components such as a keyboard 19 and/or mouse 20, a joystick or foot pedal. The example shown in FIG. 3 provides a number of treatment options. The first option is complete control of the treatment by the laser light application means 15. With this option, laser light application means receives data about the treatment from the reference data receiving means, and controls the output of the laser light source 12 employing tracking discussed below, together with the laser positioning means 14 to apply laser light to the retina of the eye 3. The laser light application means 15 is adapted, using appropriate real time image recognition discussed in the manner below, in combination with current retina position detecting means 15 to monitor the position of light from the laser light source relative to features on the retina of the eye, and to track the retina's position accordingly.

This example also enables a semi-automated process, in which particular points or areas on the retina are selected for treatment by an operator selecting points or areas displayed on the monitor 18. Once selection has been completed, the apparatus then proceeds to treat the selected points or areas by application of the required level of laser light from the laser light source 12 whilst tracking the eye.

FIG. 4 shows the optional diffractive optical element 16 shown in FIG. 2. This light diffractive element 16 diffracts a single laser beam 30 into a laser beam array 31 which, in this case, is a three-by-three matrix. It will be appreciated that matrices of other sizes could also be produced. The provision of this diffractive optical element 16 enables the application of laser light to multiple points in a single application, speeding up the treatment process, and reducing discomfort to the patient. As can be seen from FIG. 5, with a three-by-three diffractive optical element 16, the application of eight separate pulses, providing eight arrays of application points A to H, which can cover a large area of the retina 3 when the diffractive optical element 16 is provided.

The apparatus may incorporate a laser control algorithm which spatially and or temporarily randomises or disperses application of light to selected treatment parts.

FIG. 6 shows an example of one image recognition process that may be used in conjunction with the apparatus of the present invention. This process, performed by the computer 15, employs the outline of the optic nerve as a reference point on the retina (although an alternative reference may be employed by an operator), and searches through the output of the video camera 17 until a match is produced. The current retina position detecting means 15 can then track the position of the retina 3 through both voluntary and involuntary eye movements by locking into and following the optic nerve position, or another topographical feature of the retinal image such as a blood vessel, for example. The process may be employed with the invention, or may be employed in any combined retina viewing device employing laser treatment and appropriate image capturing, processing and laser control circuitry. The process of this example employs a cross-correlation function of the type

$${R}_{f t} ( u , v ) = {\sum }_{x = - M}^{M} {\sum }_{y = - N}^{N} f ( x , y ) \cdot t ( x - u , y - v )$$

where an image F(F,Y) and template T(Y), are captured and generated respectively. It will be appreciated that alternative functions may be employed.

This function is at a maximum when a portion of image F which is under template T as exactly the same as T. This can then form a simple goodness-fit measure, having a range from 0 to 1 by computing R_{ft} (u, v) /Rₜₜ(u,v).

Where Rₜₜ is computed once, before any matching has to be done.

The deviation of the current captured image from a reference image (and its corresponding template) yields a number in X and Y dimensions which is essentially an estimate of deviation. This number can then be employed to drive laser light deflecting means to correctly align the laser beam to a selected target.

This process is particularly useful if an automated or pre-stored treatment data course of treatment is required.

It will be appreciated that the overall control of the present invention can be provided by any suitable computer or microprocessor based system. For example a PC, Apple MacIntosh or other work station system with appropriate software and control interfaces. This means that control of the system can be realised with high reliability and at relatively low cost.

1. An apparatus for applying laser light to at least one selected location on the retina of the eye, the apparatus comprising:
retina image obtaining means for obtaining the image of a retina;
retina image display means for displaying the required retina image;
reference data receiving means for receiving, from an operator, data relating to a treatment to be performed;
template generating means for generating a reference template on the retina image;
target position receiving means for receiving data related to at least one target point to which light is to be applied based upon the received reference data;
current retina position detecting means for acquiring a current retina image from the retina image acquisition means and comparing it to the generated template and said reference data and outputting a signal indicating current retina position within said template; and
laser light application means for directing and applying laser light to a retina dependent upon the output of the current retina position detecting means and the target position receiving means.

2. An apparatus according to claim 1, wherein the reference data receiving means receives data relating to at least one reference point selected by an operator.

3. An apparatus according to claims 1 or 2, wherein the relationship between the generated template and the current retina position is obtained by the current retina position means by employing a confidence level algorithm.

4. An apparatus according to claim 3, wherein the confidence level algorithm employs a cross-correlation function.

5. An apparatus according to any of the preceeding claims, further including a laser light output inhibiting means for inhibiting laser light output if the detected current retina position cannot be aligned with the template.

6. An apparatus according to any of the preceeding claims, further including a laser light output inhibiting means for inhibiting laser light output if an error in the apparatus is detected.

7. An apparatus according to any of the preceding claims, which can be attached to a viewing apparatus.

8. An apparatus according to claim 7, wherein an operator views the position of low level laser light emitted by a laser light source by viewing the retina through an eye piece of the viewing apparatus.

9. An apparatus according to claims 7 or 8, wherein the viewing apparatus comprises a fundus camera.

10. An apparatus according to claims 7 or 8, wherein the viewing apparatus comprises a slit lamp.

11. An apparatus according to claim 7 or claim 8, wherein a video camera is attached to the output of the viewing apparatus and the video camera output is displayed on a video monitor.

12. An apparatus according to any of the preceding claims, wherein points on the retina may be selected for laser treatment by a user.

13. An apparatus according to claim 12, wherein the points are selected by means of a mouse or trackball with an output connected to the reference data receiving means.

14. An apparatus according to any of the preceding claims, further comprising a diffractive optical element placed within the optical path of the laser light application means, such that, from a single primary laser beam, an array of secondary treatment beams are produced.

15. An apparatus according to any of the preceeding claims, wherein the laser light is from a tube laser.

16. An apparatus according to any of claims 1 to 14, wherein the laser light is from a solid state laser.

17. An apparatus according to any of claims 1 to 14, wherein the laser light is from a diode laser.

18. An apparatus according to any of the preceeding claims, wherein the laser light application means employs a galvanometer and mirror arrangement.

19. An apparatus according to any the preceeding claims, wherein the laser light application means includes acousto- optical devices.

20. An apparatus according to any of the preceeding claims wherein the laser light application means is employed to modulate the light emitted by the laser light source by directing it away from the eye in a pulsed manner.

21. An apparatus according to any of claims 1 to 8, wherein the laser light source is pulsed.
